(19) United States
(12) Patent Application Publication
(10) Pub. No.: US 2014/0378858 A1
(43) Pub. Date: Dec. 25, 2014

(54) Apparatus And Method For The Mobile Determination Of A Physiological Stress Threshold Value

(71) Applicant: adidas AG, Herzogenaurach (DE)

(72) Inventors: Roger Armitage, Cirencester (GB); Christian Heyde, Freiburg (DE)

(21) Appl. No.: 14/310,706

(22) Filed: Jun. 20, 2014

(30) Foreign Application Priority Data
Jun. 24, 2013 (DE) 10 2013 211 908.9

(51) Int. Cl. A61B 5/00; A61B 5/08; A61B 5/091

(52) U.S. Cl. CPC A61B 5/6804; A61B 5/091; A61B 5/0806; A61B 5/4884; A61B 5/7282
USPC 600/538

(57) ABSTRACT

An apparatus for the mobile determination of at least one physiological stress threshold value of an athlete. The apparatus includes a sensor for determining the respired air volume at each point in time of a plurality of points in time. A processing unit is configured to compute a sum value for each point in time of the plurality of points in time at least based on the respired air volume of a present point in time and a sum value of a previous point in time, to set the sum value to an initial value, if the previous point in time is not within the plurality of points in time, and to determine the physiological stress threshold value based on the computed sum values.

### TECHNICAL FIELD

Embodiments of the present invention relate to an apparatus and a method for the mobile determination of at least one physiological stress threshold value of a person.

### BACKGROUND

Physiological stress threshold values deliver important information about the fitness, i.e., the training state, of an athlete. Physiological stress threshold values are usually defined as a prominent change of certain physiological parameters like, for example, the concentration of lactate in the blood (lactic acid) or the concentration ratio of oxygen and carbon dioxide in the exhaled air volume of the athlete with increasing stress intensity. It is assumed that the change in these physiological parameters is combined with a change in energy supply of the muscles of the athlete.

Based on lactate-performance diagnostics, per definition, only a physiologically relevant stress threshold value can be determined at the first significant rise of the lactate performance curve (Lactate Threshold, LT) (Beaver et al., 1985). Due to mathematical difficulties in determining the LT, in the past, a more accurately determinable and reproducible reference point (Individual Anaerobic Threshold, IAT) in the steeper course of the lactate performance curve to the right of the LT was looked for (Röcker et al., 1998, and Dickhuth et al., 1999).

By the respiratory gas analysis, a first disproportionate rise of the exhaled carbon dioxide compared to the inhaled oxygen (Anaerobic Threshold, AT) can be detected (Beaver et al., 1986). It is assumed that the first disproportionate rise of the exhaled carbon dioxide results as a direct consequence from the buffering of blood lactate (through bicarbonate) and that hence there is a causal link between LT and AT. A further disproportionate rise of minute ventilation as compared to a constant amount of carbon dioxide of the exhaled air is defined as Respiratory Compensation Point (RCP). This point represents the onset of an inadequate hyperventilation as a consequence of increasing acidosis, caused, inter alia, by the increase of blood lactate concentration.

During a stress below the AT, different macronutrients such as sugar (carbohydrates) fatty acids and proteins (amino acid) are primarily metabolized using oxygen. This way of providing energy is also denoted as anaerobic and serves to resynthesize adenosine triphosphate (ATP). ATP is the energy carrier which causes the contraction of the muscle. The energy needed for the muscle contraction is for the most part provided by hydrolysis (absorption of water) of ATP in adenosine diphosphate (ADP) and phosphate. An endurance performance below the AT can be maintained for a long time, e.g., during a marathon run.

During a stress above the AT, the aerobic energy supply is increasingly supported by anaerobic (i.e., primarily without oxygen) ways of energy supply. In this process, during the forced decomposition of dextrose (glucose) or glycogen (a form of storage of glucose) is transformed into lactate in glycolysis pyruvic acid. This happens when the pyruvic acid and oxygen processing capacities in the mitochondria are reached or the oxygen supply by the blood is limited. The resulting lactate can be retransformed into pyruvic acid by surrounding muscle fibers with still available capacities and then be metabolized in the mitochondrion over the aerobic way of energy supply. In addition, the resulting lactate is introduced from the muscle into the blood circulation and metabolized in the skeletal muscles, the heart or the brain with free capacities under aerobic conditions or converted back into glucose in the liver.

With increasing stress intensity, more and more lactate passes into the blood, so that an acidosis (hyperacidity) of the whole organism occurs in the further progress. Buffer mechanisms (alkaline counter-measure) counteract the dropping pH-value. The beginning hyperventilation after the RCP is of particular importance, since it represents, in its functionality, the upper limit of all buffer mechanisms in the organism.

The endurance performance thus does not only depend on the AT and the availability of substrate (full energy storage), but also, depending on the intensity, on the acidity tolerance and the buffer capacity. Also during longer competitions, the capacity of providing, for a short time and temporarily, significantly more energy by means of anaerobic metabolism plays an important role in specific competition situations, e.g., with sprints during a football (soccer) match.

In the course of developing numerous threshold models, in the German and English-speaking areas, different designations both for ventilatory and lactate thresholds were used which are often confounded with regard to their importance. Innumerable determination methods additionally led to confusion. Generally, in lactate diagnosis, the determination of the IAT by calculating the minimum lactate equivalent (also called basic lactate) and an added fixed amount proved to be very practical for evaluating the endurance performance of different athletes and normal persons (Ricker et al., 1998).

The minimum lactate equivalent (lactate/performance) serves as a mathematic tool for determining the first rise of blood lactate concentration (LT). In the German-speaking area, the LT is also denoted as anaerobic threshold. The LAT calculated therefrom is merely a reference point in the steeper section of the lactate performance curve which shows a higher reproducibility than the LT (Dickhuth et al., 1999).

On the basis of previous investigations, it is assumed that the LT (from lactate diagnostics) and the AT (from the respiratory gas analysis) are in causal connection and occur at approximately the same time (AT somewhat delayed). In contrast to lactate diagnostics, from the respiratory gas analysis, the RCP may, in addition, be calculated which, in turn, is in high correlation to IAT (Dickhuth et al., 1999). Compared with AT, the RCP is by far easier to calculate. Since the AT and the RCP are calculated from the respiratory analysis, these thresholds are often also denoted as ventilatory or respiratory thresholds.

From the information on stress intensity at the LT and the stress intensity at the RCP, the relative functional buffer capacity (RFBC) can be calculated. This provides an individual evaluation of the quality of available compensation mechanisms against the pH-decline caused by a stress acidosis.

The determination of physiological stress threshold values known in the prior art is disadvantageous in that it limits the athlete considerably because it is virtually only possible under laboratory conditions. Thus, the determination of the lactate concentration requires a regular (e.g., every three minutes) taking of blood, for example, at the earlobe during increasing stress intensity, e.g., on a treadmill or an ergometer.

The determination of the ventilator threshold requires the measurement of the respiration gases by means of a spirometer (aeroplethysmorgaph) which analyzes the exhaled air of the athlete. The stress control is often performed by means of an ergometer. During the measurement, the athlete wears a face mask to which a volume sensor for measuring the respired air volume as well as a hose, the so-called suction line, are connected. A part of the exhaled air is guided via the suction line to gas sensors in the aeroplethysmograph where its gas content is analyzed.

The known methods for determining physiological stress threshold values can, therefore, virtually only be performed in a laboratory. The athlete is bound to certain devices like an ergometer or a treadmill and may not freely move. Furthermore, special and complex laboratory equipment is needed for determining the lactate concentration and the respired gas concentration.

Therefore, it is the objective of the present invention to provide an apparatus for determining a physiological stress threshold value which is mobile, i.e., which may be carried by the athlete without noteworthy limitation during the exercise of almost any sports activity. The apparatus should furthermore be inexpensive to be used in popular sports. A further aspect of the present invention concerns a corresponding method.

### BRIEF SUMMARY

According to a first aspect of the present invention, this objective is solved by an apparatus for the mobile determination of at least one physiological stress threshold value of an athlete, wherein the apparatus comprises a sensor for determining the respired air volume at each point in time of a plurality of points in time, and a processing unit, which is configured to compute a sum value for each point in time of the plurality of points in time at least based on the respired air volume of a present point in time and the sum value of a previous point in time, to set the sum value to an initial value, if the previous point in time is not within the plurality of points in time, and to determine the physiological stress threshold value based on the computed sum values.

The apparatus according to the invention has the advantage as compared to known apparatuses that only a sensor for determining the respired, i.e., exhaled and/or inhaled air volume and a processing unit are needed. A regular taking of blood for measuring the lactate concentration or gas sensors for measuring the concentration of oxygen and carbon dioxide in the respired air can be done without. The apparatus is thus mobile and may be worn on the body by the athlete, for example.

In the apparatus according to certain embodiments, a sensor determines the respired air volume of the athlete at each point in time of a plurality of points in time. The respired air volume may be the inhaled or exhaled air volume or a value derived from both volumes. A point in time may in the context of certain embodiments also be a time period during which the respired air volume is determined, for example, a time period of ten seconds. The respired air volume could, for example, be averaged over this time period.

The determination of the physiological stress threshold value from the respired air volumes determined by the sensor is made possible by the processing unit according to certain embodiments. This unit measures the respired air volume at certain points in time and computes sum values for each point in time at least based on the respired air volume of a present point in time and the sum value of a previous point in time. By this kind of summation of timely subsequent air volumes, the smallest changes in the respired air volume may be determined and one or more physiological stress threshold values may be determined.

It is a particular advantage of certain embodiments that the data required for determining the physiological stress thresholds can be calculated independently of any laboratory. In contrast to known methods, the athlete is, for example, not bound to an ergometer or has his blood to be taken at regular intervals. Instead, the athlete may exercise almost any sports activity during the recording of the data.

Finally, due to the apparatus according to certain embodiments, complex and expensive laboratory equipment is not necessary. The sensor used according to certain embodiments can also record data in real-time and thus be used during normal sports activities, for example. Suitable sensors for determining the respired air volume are, for example, described in WO 2010 027515 A1 and WO 2006 034291 A2 of Vivometrics Inc. and may be integrated in sports apparel.

In one embodiment, the apparatus is configured to be worn during a sports activity of the athlete without essentially limiting the athlete in his activity. “Essentially” means that the athlete may perform his sports activity as he is used to, as if he were not carrying the apparatus. With the known determination of, e.g., the blood lactate value, a runner, for example, may not run an arbitrary track alone, but needs a third person (e.g., a sport medicine specialist) regularly taking his blood. Therefore, the blood lactate value is usually measured in a laboratory on a treadmill or ergometer. When measuring the oxygen and carbon dioxide concentration in the respired air, the athlete must wear a face mask, whereby he is essentially limited in his sports activity and not mobile.

In one embodiment, the sensor is based on the principle of respiratory inductive plethysmography. With this method, the measuring of the respired air volume is performed through meander-shaped electrical conductors which can be arranged in the chest and/or abdomen region and respectively form an electrical coil and are connected to an electrical oscillator. Due to the respiratory motions, the circumference of the chest and abdomen and thus the length of the conductors, the inductance of the coils and finally the oscillation frequency are altered. The alteration of the oscillation frequency can be evaluated and permits conclusions to be made with regard to the inhaled and exhaled air volume. Corresponding sensors are, for example, described in the mentioned WO 2006 034291 A2.

Alternatively, the respired air volume can also be determined by means of a magnetometer or a pressure sensor. A further alternative is the determination of changes in length in the thoracic region. In principle, an optical sensor can also be used for this. The respired air volume can also be determined indirectly by the corresponding processing of other measured values. However, according to certain embodiments, any sensor can be used which is capable of determining the respired air volume.

In one embodiment, the sensor is integrated in sports apparel. In one embodiment, in addition or alternatively, the processing unit is integrated in an article of sportswear. For example, a sensor based on the principle of respiratory inductive plethysmography may be sewn, woven or glued into a running shirt, soccer or bicycle jersey. The athlete does not have to don the sensor and/or processing unit in addition to his sports apparel or fix the sensor and/or processing unit thereon. Also, a sensor integrated in the sports apparel is not so likely to slip out of place and delivers more reliable measurement results.

In one embodiment, the sensor and the processing unit are connected with each other via at least one electrical link. This may be a cable or an electrically conductive fabric. Alternatively, the sensor and the processing unit are wirelessly connected with each other.

In one embodiment, the processing unit is a processor. A processor may easily and relatively inexpensively be programmed for determining the physiological stress threshold value or may be configured correspondingly in another way.

In one embodiment, the processing unit may be worn together with the sensor on the body of the athlete, for example, in a sports shirt or an electronic apparatus taken along as, for example, a mobile telephone or a media player. Alternatively, the processing unit may be separated from the athlete and communicate wirelessly, for example, with the sensor. For example, the processing unit might be a personal computer, a notebook or a tablet PC which is monitored by a trainer of the athlete, for example.

In one embodiment, the apparatus comprises an alarm device for outputting an alarm if the determined physiological stress threshold value is reached and/or exceeded. In this way, the athlete is warned if an intensity of stress is reached which does not correspond to his training goal. Depending on the training goal, ventilator stress thresholds are an objective measurement for training control. For example, training above the anaerobic or ventilatory threshold, respectively, is not optimal for endurance sports and fat burning. The athlete may adapt his stress level accordingly if an alarm occurs. For example, a runner may reduce his speed.

In one embodiment, the processing unit is configured to multiply the determined air volumes by times associated with the respective points in time. Such a method step leads to a smoothing of the natural spread of the calculated air volumes and thus to a more reliable prediction. In one embodiment, the zero-point of time is associated with the earliest point in time of the plurality of points in time.

In one embodiment, the sum includes a difference which is based on the respired air volume of the present point in time and the respired air volume of a previous point in time. This method step allows the sensitive detection of the smallest changes in the slope of the respired air volume determined over time.

In one embodiment, in each sum a weight value is subtracted from the respective determined sum value. A correspondingly chosen weight value ensures that changes in the slope of the determined air volumes are prominently visible, such that the physiological stress threshold value may be determined most reliably.

In one embodiment, the weight value is constant in time during the plurality of points in time. For example, the weight value may be the expected average value of the respired air volumes such that deviations from the expected average show up in a particularly prominent manner.

In one embodiment, the weight value is based on an average value over the plurality of points in time. Temporary deviations from the average value, e.g., when reaching a physiological stress threshold value, are thus recognized most reliably.

Alternatively, the weight value is the respired air volume of the first point in time. Furthermore, alternatively, the weight value is an average value to be expected. For example, it might be the expected average value of a statistical population of athletes.

In one embodiment, the average value is based on the respired air volumes. Air volumes which temporarily exceed or fall below the average air volume, e.g., when reaching a physiological threshold, may therefore be determined very reliably due to the applied summation method.

In one embodiment, the processing unit is configured to determine the physiological stress threshold value based on a change over time of the sum values. A change over time of the sum values computed according to certain embodiments is a reliable indicator for reaching a physiological stress threshold value.

In one embodiment, the processing unit is configured to determine the physiological stress threshold value based on at least one regression line. Regression lines allow for the modeling of a trend in the computed sum values. A change in the underlying trend may be used as an indication for reaching a physiological stress threshold value.

In one embodiment, the processing unit is configured to determine the physiological stress threshold value based on a point of intersection of two regression lines. In this way, a change in the underlying trend and thus the presence of a physiological stress threshold value may be reliably determined.

In one embodiment, the physiological stress threshold value is one of the ventilatory thresholds. As already mentioned, the second ventilator threshold (RCP) strongly correlates with the IAT. The respiratory compensation point RCP (also designated as respiratory threshold) is above the AT (Anaerobic Threshold) or the LT (Lactate Threshold). According to a common definition, the respiratory compensation point denotes a disproportional rise in minute ventilation compared to the steadily increasing release of carbon dioxide. According to another definition, the respiratory compensation point denotes the point where, with increasing stress of the body, a decrease of carbon dioxide concentration in the respired air can be determined.

Alternatively, based on a corresponding y-axial intercept, at the determined stress threshold value, a further stress threshold value can be determined in the evolution of the ventilation data over time, which is around a relative fixed value (e.g., 75%) before the calculated stress threshold value. Said stress threshold value estimates the situation of AT or LT, respectively.

Alternatively, the relative span between the stress threshold values is an estimate of the relative functional buffer capacity RFBC which is relevant for sports with high intermittent stress intensities such as game sports.

The presence of at least one stress threshold value constitutes important information for the athlete. On the one hand, the athlete may determine his stress threshold values by means of the apparatus according to certain embodiments (for example, LT or AT, respectively, and IAT and RCP, respectively), determine competition prediction for standardized running routes and compare his performance capacity to that of a comparative population. On the other hand, the apparatus according to certain embodiments may be used for training control, i.e., it may inform the athlete when the individual ventilator threshold or AT, respectively, is reached during a training session, and/or give the athlete an opportunity to check his training success on his own.

The stress thresholds are at a higher stress intensity with trained athletes than with untrained athletes. Therefore, the respiratory compensation point gives an important hint to the training state of an athlete and may therefore be used for training control.

In one embodiment, the apparatus is configured to determine the at least one physiological stress threshold value in real-time. Thus, the athlete may obtain a feedback during the training relating to his stress intensity and may react when the physiological threshold is exceeded.

A further aspect of the present invention relates to a method for the mobile determination of at least one physiological stress threshold value of an athlete, comprising the steps: determining the respired air volume at each point in time of a plurality of points in time; computing a sum value for each point in time of the plurality of points in time at least based on the respired air volume of a present point in time and a sum value of a previous point in time; setting the sum value to an initial value, if the previous point in time is not within the plurality of points in time; and determining the physiological stress threshold value based on the computed sum values.

### BRIEF DESCRIPTION OF THE DRAWINGS

The following aspects of certain embodiments are described in more detail referring to the accompanying figures.

FIG. 1: A schematic illustration of an apparatus according to an embodiment, which may be used with sports apparel.

FIG. 2: A diagram, which shows the time evolution of sum values computed from the respired air volume according to an embodiment.

FIG. 3: A graph produced according to an embodiment of the present invention.

FIG. 4: A comparison of competition predictions according to an embodiment of the present invention with competition predictions based on laboratory values.

### DETAILED DESCRIPTION

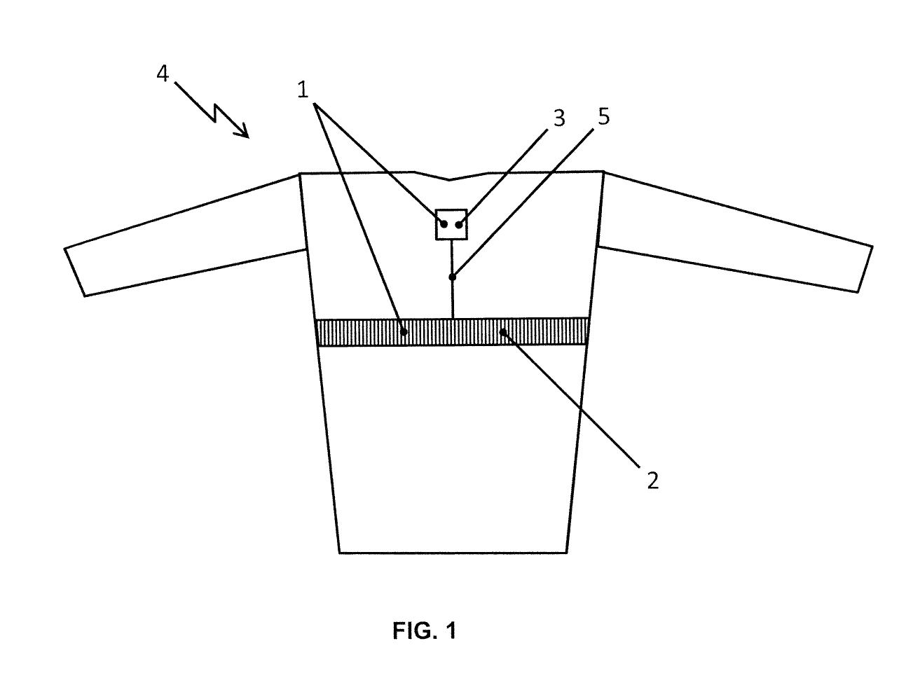

FIG. 1 shows one embodiment of the present invention. In FIG. 1 an apparatus 1 according to an embodiment is shown for the mobile determination of at least one physiological stress threshold value of an athlete. The apparatus 1 comprises a sensor 2 for determining the respired air volume at each point in time of a plurality of points in time. The sensor 2 is integrated in sports apparel 4 in the embodiment of FIG. 1. In certain embodiments this is a t-shirt, e.g., a running shirt, soccer or bicycle jersey. The sensor 2 may be separate from the sports apparel in certain embodiments.

The sensor 2 may be a sensor which is based on the principle of respiratory inductive plethysmography. With this method, the measuring of the respired air volume is performed through meander-shaped electrical conductors which can be arranged in the chest and/or abdomen region and respectively form an electrical coil and are connected to an electrical oscillator. Due to the respiratory motions, the circumference of the chest and abdomen alter and thus the length of the conductors, the inductance of the coils and finally the oscillation frequency. The alteration of the oscillation frequency can be evaluated and permits conclusions to be made with regard to the inhaled and exhaled air volume. Corresponding sensors are, for example, described in previously mentioned WO 2006 034291 A2.

The sensor 2 may be based on another principle of measuring the respired, i.e., the inhaled and/or exhaled air volume. For example, it may be an aeroplethysmograph.

The sensor 2 is configured such that the respired air volume is determined at each point in time of the plurality of points in time. The respired air volume may be the inhaled air volume or the exhaled air volume. Alternatively, the sensor 2 determines the inhaled air volume as well as the exhaled air volume. In this case, it could, for example, determine the inhaled air volume as the average value of the inhaled and exhaled air volume.

The plurality of points in time are at least two different points in time. A point in time is understood in the context of the present invention also as a time period, for example, a time period of ten seconds. In this case, the point in time may be defined, for example, as the start point or the end point or the middle point of the time period.

The sensor 2 may determine the respired air volume breath by breath. In this case, a point in time is assigned to each breath, where the breath occurred. Since human respiration usually is somewhat irregular, the time differences between the points in time are irregular as well. Alternatively, the sensor 2 may determine the respired air volume for consecutive points in time. For example, the respired air volume may be determined over a time period of ten seconds each. In this case, the time differences between the points in time are the same.

In one embodiment, the apparatus comprises a processing unit 3, which in the exemplary embodiment of FIG. 1 is fixed to the sports apparel 4. For example, the processing unit 3 may be integrated in the sports apparel, e.g., sewn or glued. Alternatively, the processing unit 3 may be releasably attached to the sports apparel 4, for example, by means of at least one push-button or hook-and-loop fastener.

In the exemplary embodiment of FIG. 1 the processing unit 3 is arranged in the upper back portion of the sports apparel 4. The processing unit 3 may be arranged at an arbitrary location of the sports apparel or on the body of the athlete.

The processing unit 3 may, for example, be a processor which is programmed by suitable software. Alternatively, the functions performed by the processing unit 3 are directly integrated in hardware, for example, as an application-specific integrated circuit (ASIC) as a field programmable gate array (FPGA).

In one embodiment the apparatus 1 is suitable to be worn during a sports activity of the athlete without essentially limiting the athlete in his activity. “Essentially” means that the athlete may perform his sports activity as he is used to, as if he were not carrying the apparatus 1. For example, the athlete may wear the sports apparel 4 in the exemplary embodiment of FIG. 1 like any other sports apparel, without being limited in his motions.

The processing unit 3 may be separated from sports apparel 4. For example, the processing unit 3 may comprise a wrist band and may be worn around a wrist or an upper arm of an athlete. Alternatively, the processing unit 3 may be worn on a belt and may be attached thereto, e.g., by a clip. It is furthermore conceivable that the processing unit 3 is integrally formed with the sensor 2.

In the exemplary embodiment of FIG. 1, the processing unit 3 is connected to the sensor 2 by means of an electrical conductor 5. The electrical conductor 5 may, e.g., be integrated in the sports apparel 4 via the electrical conductor 5. The sensor 2 transmits a signal to the processing unit 3 which corresponds to the respired air volume as determined by the sensor 2.

Alternatively, the sensor 2 transmits a signal to the processing unit 3 wirelessly, for example, by radio. It is conceivable to use Bluetooth or another protocol.

The processing unit uses the air volumes as determined by the sensor 2 to compute a sum value for each point in time of a plurality of points in time, at least based on the respired air volume of a present point in time and a sum value of a previous point in time. For example, the processing unit 3 determines a sum value Sₙ for a present point in time n with the sum:

Sₙ=Sₙ₋₁+vₙ

Here, Sₙ₋₁ is the previous sum value, i.e., the sum value computed at the previous point in time n−1 and vₙ is the respired air volume of the present point in time n, i.e., the respired air volume which is determined by the sensor 2 at the present point in time.

Furthermore, the processing unit 3 is configured to set the sum value to an initial value if the previous point in time is not within the plurality of points in time. For example, if the plurality of points in time comprises the points in time (1, 2, . . . , N), the processing unit 3 sets the sum value zero to an initial value a:

S₀=a

The next sum value S₁ computed by the processing unit 3 is then:

S₁=S₀+x₁=a+x₁

The initial value may, for example, be zero: a=0. In one embodiment, a weight value wₙ is subtracted from each determined sum value:

Sₙ=Sₙ₋₁+vₙ−wₙ with S₀=a

The weight value may be constant over the plurality of points in time.

w≡w (for all n from the plurality of points in time).

The weight value wₙ may be constant in part, i.e., over sections of the plurality of points in time. For example, the weight value in the anaerobic area could take a determined constant, whereas in the anaerobic area, it takes a different constant value.

In the case of a constant weight value, it may be an average value which is computed over the plurality of points in time. In one embodiment of the invention, the average value is based on respired air volumes. The processing unit 3 is configured to compute the average air volume v̅ for each point in time of the plurality of points in time:

$${w}_{n} ≡ w = \overline{v} = \frac{1}{N} {\sum }_{n = 1}^{N} {v}_{n}$$

Here N is the number of points in time of the plurality of points in time. The processing unit 3 is, therefore, configured in this embodiment to compute the sum values for the point in time n according to the following formula:

Sₙ=Sₙ₋₁+vₙ− v̅ with S₀=0

In this case, it becomes clear that physiological stress threshold values show up very prominently and may be identified very reliably.

Alternatively, the weight value may, for example, be set on the first obtained measurement value: wₙ≡x₁. Other than with the use of the average value v̅ as weight value, in this case, not all measurement values have to be known. The process may then be performed in real-time.

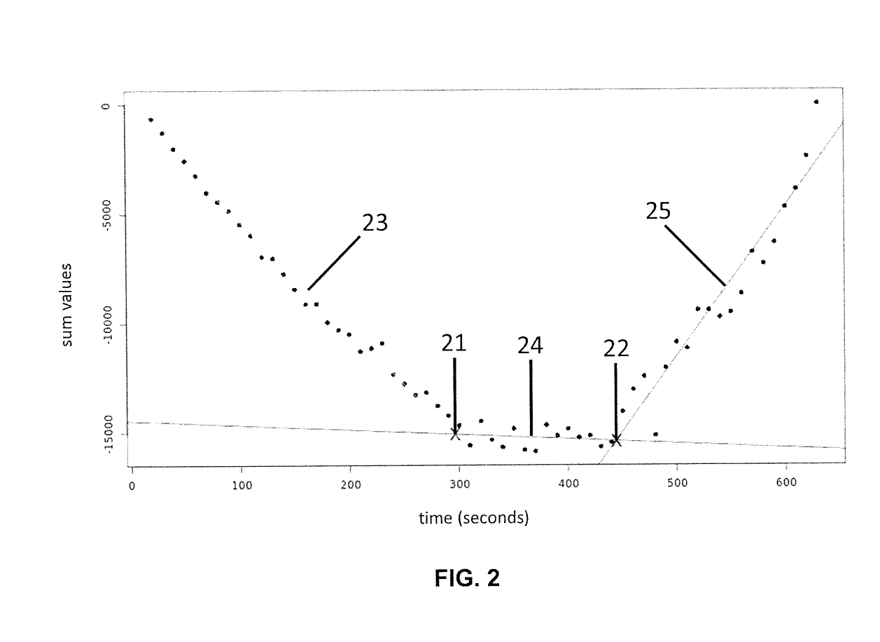

FIG. 2 shows an exemplary diagram in which the processing unit 3 has computed the sum value according to the above formula Sₙ=Sₙ₋₁+vₙ− v̅. In the diagram of FIG. 2, the sum values computed according to the above formula are plotted against the time (in seconds). The respired air volumes were averaged over time periods of ten seconds each. The initial value was set to zero: a=0. Since the average respired air volume over the plurality of points in time has been subtracted in each summation, the last computed sum value is zero as well: Sₙ=0.

As can be seen from the diagram of FIG. 2, a kind of summation according to the formula above emphasizes temporary deviations from the average respired air volume. Thus, between 0 and 300 seconds, a negative trend is visible, i.e., in this time span the respired air volume was smaller on average than the average air volume v̅ respired per point in time over the entire time span of about 600 seconds (i.e., the plurality of points in time).

During the time span of about 300 seconds to 450 seconds, a flat evolution of the computed sum values shows up. In this time span the respired air volume was approximately equal to the average value v̅. From about 450 seconds on, the respired air volume is higher than the average value v̅.

Summarizing, it results from the diagram of FIG. 2 that the respired air volume for each point in time has increased over the entire measured time span, i.e., the plurality of points in time. This is in accordance with the continuously increasing stress intensity during the entire time span. To compensate for the increased need of oxygen for the energy supply of the muscles and to remove the generated carbon monoxide, the air volume respired per point in time has increased.

As a result of the diagram of FIG. 2 and as will be explained in the following, this increase of the respired air volume occurs abruptly at certain physiological stress threshold values. The processing unit 3 is therefore configured to determine the at least one physiological stress threshold value based on the computed sum values.

In one embodiment, the processing unit 3 determines the physiological stress threshold value based on timely changes of the sum values. For example, in the diagram of FIG. 2, a prominent change of the computed sum values occurs at about 300 seconds. As initially explained, the respired air volume increases at this point and corresponds approximately to the average air volume v̅ of the entire time span of about 600 seconds. This point of the first prominent rise 21 of the respired air volume corresponds well with the ventilator threshold. The point of the second prominent rise 22 of the respired air volume (at about 450 seconds) corresponds well with the respiratory compensation point.

Three regression lines 23, 24, 25 are drawn in the diagram of FIG. 2. In one embodiment, the processing unit 3 uses regression lines to determine the at least one physiological stress threshold value. For example, a relation to the ventilatory threshold may be determined as the intersection 21 of the first two regression lines 23 and 24. A relation to the respiratory compensation point may be determined as the intersection 22 of the second regression line 24 and the third regression line 25.

To determine the location of the regression lines and the intersections, known algorithms may be used. As an initial parameter, such an algorithm may, for instance, comprise an area where an intersection is presumed. For example, the first intersection could be located in an area of 0% to 60% of the maximum stress of the athlete.

The apparatus 1 according to one embodiment may comprise an alarm device (not shown in FIG. 1) which outputs an alarm upon reaching and/or exceeding the determined stress threshold value. For example, this may be an acoustical or optical alarm, which outputs a corresponding alarm upon reaching the ventilator threshold 21 and/or the respirator compensation point 22. In this way, the apparatus 1 according one embodiment may be used for training control.

After determining at least one physiological threshold value according to one embodiment, in the following, this threshold may be used for training control. The athlete then no longer has to go to his stress limit. The at least one physiological stress threshold value determined according to one embodiment may be used in a training unit, for example, beside the speed and the heart rate, as a threshold value for training goal zones.

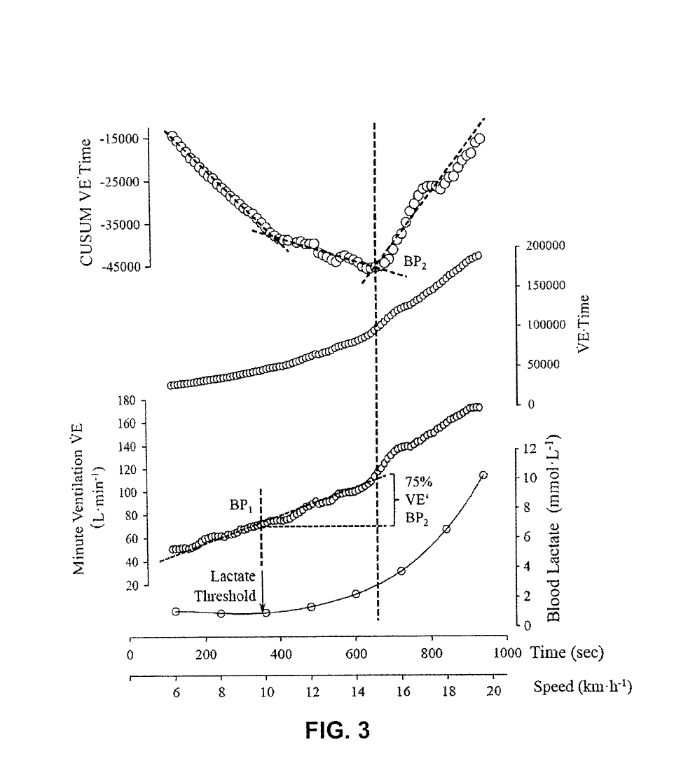

FIG. 3 shows a further possibility of using the present invention according to an embodiment. In the middle third of FIG. 3, the respired air volumes determined by means of sensor 2 are plotted against the time or the running speed, respectively, of the athlete. In the upper third of FIG. 3, the related respired air volumes processed by an apparatus according to one embodiment or by use of the method according to one embodiment, respectively, are plotted. According to one embodiment, a significant timely change of the respired air volume was determined as transition point BP₂. In the example of FIG. 3, the transition point BP₂ is located at approximately 650 seconds or at a running speed of 15 km/h.

A further point BP₁ may now be defined as a reduced relative fixed value before the determined transition point BP₂. For example, BP₁ could be defined between 50% and 100% or between 65% and 85% of the stress associated with the calculated transition point BP₂. In the example of FIG. 3, a physiological stress threshold value BP₁ is defined at 75% of the stress of BP₂. This is at approximately 350 seconds or 10 km/h, respectively, and corresponds well with the significant rise of the lactate performance curve (Lactate Threshold, LT). The lactate curve is shown in the lower third of FIG. 3 for comparison.

The apparatus according to one embodiment or the method according to one embodiment, respectively, may also be used for individual training control, for stress monitoring and for competition prediction. For example, the relative functional buffer capacity RFBC can be estimated therefor on the basis of the location of points BP₁ and BP₂:

$${RFBC}_{est} = \frac{{BP}_{2} - {BP}_{1}}{{BP}_{2}} \cdot 100$$

From the estimated relative functional buffer capacity RFBCₑₛₜ, it can, for instance, be estimated how long an athlete will need for a thousand-meter sprint. BP₁ can then—as shown in connection with FIG. 3—be determined as relative percentage stress of BP₂. Alternatively, BP₁ may be determined as the actual transition point—as shown in connection with FIG. 2.

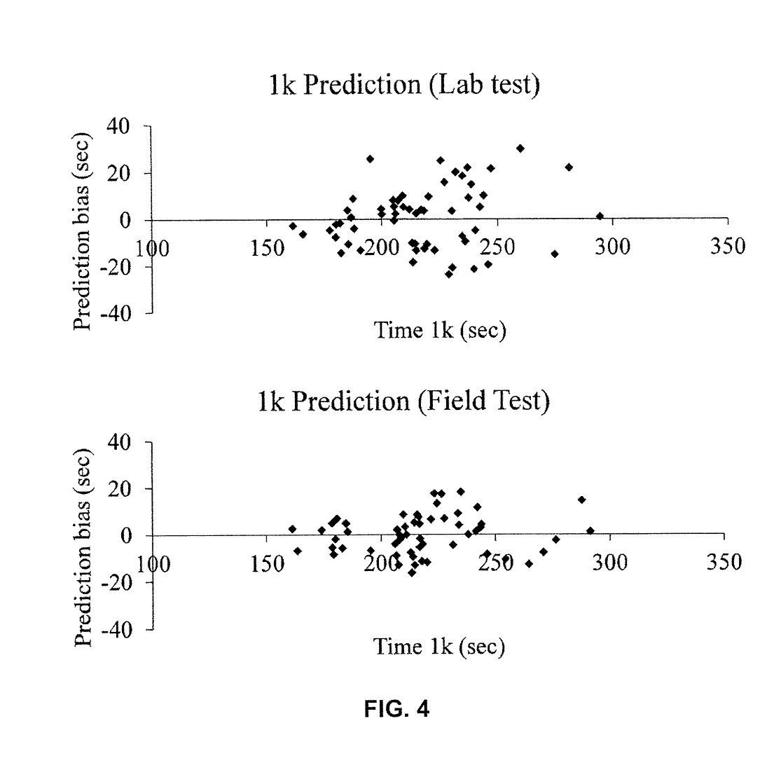

FIG. 4 shows corresponding estimates of the performance expected for a thousand-meter sprint of 59 athletes. FIG. 4 shows the deviation of the expected performance from the actual performance in seconds, i.e., the performance was defined as the time needed for a running distance of 1000 m.

For the diagram in the upper half of FIG. 4, the performance expected from each athlete was estimated in the laboratory, i.e., by means of lactate diagnostics, the IAT (Individual Anaerobic Threshold) was determined. From the IAT, the relative functional buffer capacity RFBC was calculated, and finally the time expected to be necessary for a distance of 1000 m was determined.

The diagram in the lower half of FIG. 4 is based on an apparatus according to one embodiment or a method according to one embodiment, respectively. First of all, a transition point BP₂—as shown in connection with FIG. 3—was determined. Then, BP₁ was determined as the relative fixed value and RFBCₑₛₜ was calculated as shown above. Finally, the time expected to be needed for a distance of 1000 m was determined at BP₂ and RFBCₑₛₜ on the basis of the running speed.

From a comparison of the two diagrams in FIG. 4, it becomes clear that the values calculated on the basis of certain embodiments (lower half of FIG. 4) present a significantly smaller deviation from the actual performance than the expected values determined on the basis of lactate values (upper half of FIG. 4). A statistical analysis shows an average deviation of 6.7 seconds (relative deviation 2.9%, coefficient of determination R²=0.92). In contrast thereto, the expected values determined by means of lactate values show an average deviation of 10.6 seconds (relative deviation 4.8%, coefficient of determination R²=0.86). The competition prediction determined on the basis of certain embodiments is thus significantly better than a prediction which is based on lactate values determined in the laboratory.

1. An apparatus for the mobile determination of at least one physiological stress threshold value of an athlete, comprising:
a sensor for determining the respired air volume at each point in time of a plurality of points in time; and
a processing unit, which is configured to:
compute a sum value for each point in time of the plurality of points in time at least based on the respired air volume of a present point in time and a sum value of a previous point in time;
set the sum value to an initial value, if the previous point in time is not within the plurality of points in time; and
determine the physiological stress threshold value based on the computed sum values.

2. The apparatus according to claim 1, wherein the apparatus is configured to be worn during a sports activity of the athlete without essentially limiting the athlete in his activity.

3. The apparatus according to claim 1, wherein the sensor is based on the principle of respiratory inductive plethysmography.

4. The apparatus according to claim 1, wherein the sensor is integrated in sports apparel.

5. The apparatus according to claim 1, wherein the processing unit is integrated in sports apparel.

6. The apparatus according to claim 1, wherein the processing unit is a processor.

7. The apparatus according to claim 1, further comprising:
an alarm device for outputting an alarm if the determined physiological stress threshold value is reached or exceeded.

8. The apparatus according to claim 1, wherein the processing unit is configured to multiply the determined air volumes with times associated with the respective points in time.

9. The apparatus according to claim 8, wherein the zero-point of time is associated with the earliest point in time of the plurality of points in time.

10. The apparatus according to claim 1, wherein the sum is based on a difference which is based on the respired air volume of the present point in time and the respired air volume of a previous point in time.

11. The apparatus according to claim 1, wherein in each sum a weight value is subtracted from the respective determined sum value.

12. The apparatus according to claim 11, wherein the weight value is constant in time over the plurality of points in time.

13. The apparatus according to claim 11, wherein the weight value is based on an average value formed over the plurality of points in time.

14. The apparatus according to claim 13, wherein the average value is based on the respired air volumes.

15. The apparatus according to claim 1, wherein the processing unit is configured to determine the physiological stress threshold value based on a change over time of the sum values.

16. The apparatus according to claim 1, wherein the processing unit is configured to determine the physiological stress threshold value based on at least one regression line.

17. The apparatus according to claim 1, wherein the processing unit is configured to determine the physiological stress threshold value based on a point of intersection of two regression lines.

18. The apparatus according to claim 1, wherein the physiological stress threshold value is the ventilatory threshold.

19. The apparatus according to claim 1, wherein the physiological stress threshold value is the respiratory compensation point.

20. The apparatus according to claim 1, wherein the apparatus is configured to determine the at least one physiological stress threshold value in real-time.

21. The apparatus according to claim 12, wherein the weight value is based on an average value formed over the plurality of points in time.

22. The apparatus according to claim 21, wherein the average value is based on the respired air volumes.

23. A method for the mobile determination of at least one physiological stress threshold value of an athlete, comprising:
determining the respired air volume at each point in time of a plurality of points in time;
computing a sum value for each point in time of the plurality of points in time at least based on the respired air volume of a present point in time and a sum value of a previous point in time;
setting the sum value to an initial value, if the previous point in time is not within the plurality of points in time; and
determining the physiological stress threshold value based on the computed sum values.
